(19) United States
(12) Patent Application Publication
(10) Pub. No.: US 2010/0269966 A1
(43) Pub. Date: Oct. 28, 2010

(54) Scented Balloon Cover and Methods of Scenting the Same

(76) Inventor: Sean W. Butler, Lafayette, IN (US)

Correspondence Address: FLIESLER MEYER LLP, 650 CALIFORNIA STREET, 14TH FLOOR, SAN FRANCISCO, CA, 94108, US

(21) Appl. No.: 12/606,323

(22) Filed: Oct. 27, 2009

Related U.S. Application Data
(60) Provisional application No. 61/172,858, filed on Apr. 27, 2009.

(51) Int. Cl. B65D 65/02; A61L 9/04

(52) U.S. Cl. 150/154; 239/6

(57) ABSTRACT

A method of scenting a balloon cover, the method including placing an inflatable scented balloon inside at least one cover, wherein a membrane wall of the balloon comprises a fragrance, inflating the balloon within the at least one cover, wherein the balloon transfers the fragrance to the at least one cover, and allowing the at least one cover to absorb the fragrance, thereby providing a scent to the at least one cover and surrounding environment. The cover is adapted to surround the scented inflatable balloon, wherein the body is made of a material that is adapted to absorb a fragrance from the balloon, and wherein the body includes a safety flap assembly adapted to allow the balloon to be placed inside the cover.

### PRIORITY CLAIM

This application claims priority under 35 U.S.C. 119(e) to U.S. Provisional Patent Application No. 61/172,858, filed Apr. 27, 2009, entitled “Scented Balloon Cover and Methods of Scenting the Same,” by Sean W. Butler (Attorney Docket No. NOVT-01006US0), which application is incorporated herein by reference.

### BACKGROUND OF THE INVENTION

A historical study of aroma chemistry makes clear the significance of fragrance throughout the ages. The flirtatious smell of wild flowers from around the world has been one of mother natures most original and enduring creations since the dawn of time. With the creation of the world's first modern perfumeries from the 12th century in Italy to the 14th century in France, people of all cultures and ages have come to continually enjoy both the natural and scientific refinement of fragrance in a variety of mediums that uplifts the human spirit, one's emotional mood and enhances the celebratory nature of everyday life. Advances in fragrance chemistry, aroma science and newly applied chemical engineering technologies have brought about a virtual plethora of natural and synthetic fragrances with new commercial fragrance delivery systems where work productivity has been scientifically shown to increase when certain chemical fragrances are added to the air ventilation systems of corporate offices. The Walt Disney Company remains a harbinger in this field of experimentation and study, as well as the newly formed global entity “The Scent Marketing Institute” of Scarsdale, N.Y.

Today, one finds fragrance chemistry aromatics in resin bowling balls, molded pieces of fragrant rubber in dolls, wax based lip balms, hand lotions, topical ink printing and lithography, spray or rub on fragrances, scented markers and a vast variety of flavor enhancing chemicals to improve the aroma and taste of various foods. Age groups from young children to senior citizens can both see and smell the constant creative and innovative methods of unique and novel scientific fragrance applications. Modern chemists continue to decode and reengineer the most basic elementals of molecular fragrance chemistry just as geneticists have decoded the human genome to further discover epigenetic triggers. Scent, fragrance and aroma have the capacity to swiftly trigger our memories and emotions. They can alter perception and mood. Inventors, fragrance designers, perfumers, and the burgeoning flux of new technical researchers in aroma science and chemical engineering around the world have turned natural chemical fragrances into chemically engineered formulations which are custom designed for very specialized purposes further utilizing innovative and novel delivery systems to release said fragrances.

Although prior art regarding scenting latex or foil balloon includes various methodologies, the longevity of the fragrance release still remains very problematic and often dooms the invention to not be commercially viable. More specifically, as a studied scientific event, scented latex inflatable balloons only emit an immediate burst of evaporative fragrance when inflated, which when measured over time, lasts only a few minutes. In other words, the consumer typically experiences little more than a several brief whiffs of scented fragrance due to the quick evaporative properties of the fragrant gas, which is quickly dispersed into the surrounding environment in less than a few minutes.

### SUMMARY OF THE INVENTION

The present invention relates to a scented balloon cover and methods for scenting the same. In certain embodiments, the balloon cover can be placed over an inflatable balloon that has been treated with a fragrance. A fragrance, as used herein, is any mixture of natural or synthetic chemically based essential or fragrant oils (e.g., flavor-type, florals and perfumes) designed to emit a pleasant smell. Further, the fragrance may include any variety of a simple or compounded multiplicity of chemicals. As a balloon is inflated, the expanding and thinning balloon membrane releases the fragrance within the balloon cover. The fragrance can be released from the balloon as a liquid, vapor or gas. The fragrance is absorbed into the balloon cover which provides an absorptive medium to hold the fragrance thereby aromatizing the cover.

In certain embodiments, the scented balloon cover includes time delay engineered features in its composition which results in longer lasting fragrant emission properties from within the cover. Given the chemical composition of a given fragrance, the aromatic emission not only lasts longer, but can also be made in varied strengths as a function of the fragrance's specialized chemical design. Embodiments of this invention will bring about a far more “value added” scented consumer product including both a new variety of pop culture and traditional printed textile motifs.

In certain embodiments, the balloon cover enhances the fragrance strength and duration of any current scented inflatable balloon by absorbing a cumulative volume of fragrance within its thick walls thereby increasing the concentration of fragrance in the textile cover itself. Any current scented inflatable latex balloon and/or other scented inflatable gas permeable membrane body can now be used as the fragrance driver to change a non-fragrant textile cover into an aromatized or scented object of amusement. Varied embodiments of this invention offer novel ways of creating a new balloon encasement medium of fragrance storage and enhancement. There are other varieties of formed and inflatable compounds which can hold a chemical fragrance including such compounds as natural or synthetic rubbers, polyurethanes with elastomers, polyvinyl chloride (“PVC”) compounds with plasticizers, varied blow-molded objects as scented shag or kick balls, or expandable foil balloons with fragrant surfaces.

As currently exists, scented or fragranced inflatable balloons can emit fragrances upon inflation. The unhindered evaporative release of the fragrance quickly disperses into the surrounding environment. Accordingly, certain embodiments of the present invention mitigates the unhindered evaporation of a fragrance by capturing and covering the scented balloon body emission during inflation with a dense, absorbent textile body. As the scented balloon body is expanded, thereby stretching and thinning out its chemically scented latex balloon membrane walls, a scientific fragrance chemical transference process begins whereby the scented inflatable balloon infuses or impregnates the custom fitted cover encasement with evaporative fragrance molecules into its physical interior absorbent matrix design. The fragrance molecules can then attach to both regular and/or micro-fiber filaments within the cover. As inflation continues, more fragrance molecules are released into the shaped material body sticking to the textile material, at which point it slowly begins to permeate the textile material of the encasement cover. In essence, the absorbent cover acts to both inhibit and retard the fragrant scented emissions of the original scented elastic rubber or elasticized fragrant hybrid plastic body. The unique chemical transference process is designed to create a slow and uniformly time-delayed dispersal of trapped fragrant absorbed liquid, vapor and/or gas, whose fragrant molecules ultimately find their way into the surrounding environment over an extended period of time.

In certain embodiments, the cover can be customized in most any way for a variety of safe consumer friendly purposes and markets. For example, one version of the aromatic cover with interior fragranced body is as a lightly scented play toy for the +10 year old to the later teen youth market in which one could play with the dry surface of the lightly scented medium with cool pop culture print designs. Another version could be one of a small four (4″) scented tear-drop balloon covered shape with an artistic floral print to accent or enhance any given bouquets unique natural fragrance(s) or to add fragrance to a silk floral bouquet. Yet another version of the present invention could be a stronger scented inflatable latex body inflated and covered with a unique thicker textile weave pattern material thereby creating a new type of aromatic advertising medium for well known brands as Disney cartoon characters or youth celebrity sensations as Hanna Montana or the Jonas Brothers, both of which have their own perfume/cologne lines. This invention serves as a strategic marketing add-on to their expanding general product lines and further acts to strengthen their brand marketing identities.

In certain embodiments, gas absorbent textile material can be used to muffle and provide a partial smothering of the scientific fragrance burst of fragrance upon the balloons inflation and absorb the fragrance therein. An unhindered instantaneous scented burst of fragrance from the inflation of a scented balloon with no absorbent cover is scientifically fleeting. This new invention captures the aromatic fragrance release from the scented balloon with a textile cover which now provides a temporary home for these once fleeting gaseous and gas emissions and releases them over longer periods of time, generally as a function of fragrance volume and strength, with most balloon aromatic cover encasements lasting a minimum of several days and not just a few minutes.

### BRIEF DESCRIPTION OF THE DRAWINGS

The accompanying drawings, which are incorporated into and constitute a part of this specification, illustrate one or more embodiments and together with the detailed description, serve to explain the principles and implementations of the invention. In the drawings:

FIG. 1 is a top perspective view of a completed balloon cover in accordance with an embodiment of the invention.

FIG. 2 is a bottom perspective view of a completed balloon cover in accordance with an embodiment of the invention.

FIG. 3 is a top view of two flaps used for creating an opening port in the balloon cover in accordance with an embodiment of the invention.

FIG. 4 is a top view of the two overlapping flaps that are sewn together as the balloon cover opening port in accordance with an embodiment of the invention.

FIG. 5 is a top view of a completed safety flap assembly in accordance with an embodiment of the invention.

FIG. 6 is a cross-sectional view of an inflated scented balloon inside the cover with resultant chemical transference in accordance with an embodiment of the invention.

### DETAILED DESCRIPTION

Embodiments are described herein in the context of a scented balloon cover and methods for scenting the same. Those of ordinary skill in the art will realize that the following detailed descriptions are illustrative only and are not intended to be in any way limiting. Other embodiments of the present invention will readily suggest themselves to those skilled persons having the benefit of this disclosure. Reference will now be made in detail to implementations of the embodiment of the present invention as illustrated in the accompanying drawings. The same reference indicators may be used throughout the drawings and the following detailed description to refer to the same or like parts.

An embodiment of the present invention is directed to a unique scented balloon cover. Another embodiment of the present invention is directed to the process that creates an aromatic textile medium utilizing a preferred unscented fitted patterned cover encasement, of either a geometric or non-geometric shape, over a scented inflatable latex balloon or other such elastic or expandable fragranced body that when inflated, aromatizes said textile cover with a gas, vapor and/or light liquid fragrance, thereby ultimately allowing a time-delayed delivery of fragrant aromatic gas into the surrounding environment.

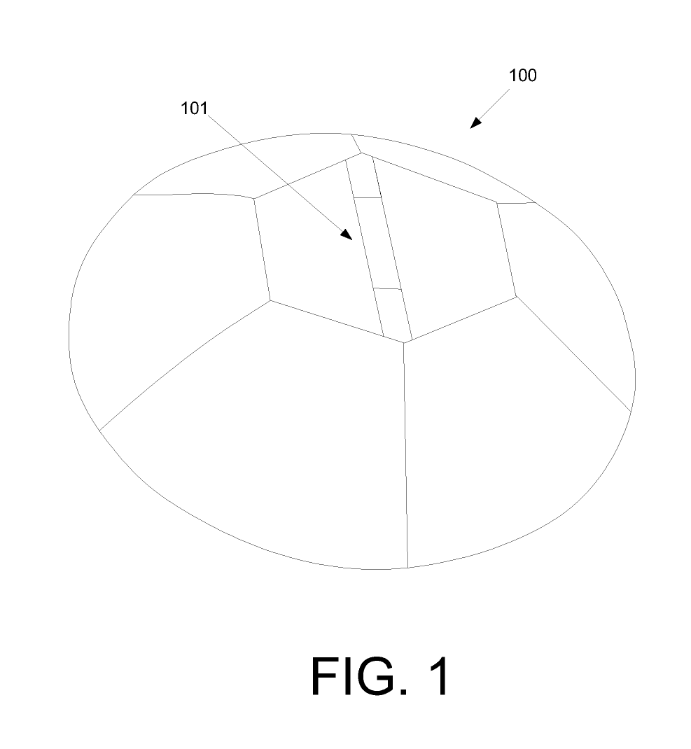

Referring now to FIG. 1, there is seen a top view perspective of an embodiment of a balloon cover, generally numbered 100, which can be manufactured as a combination of separate pieces. All the pieces can be stitched or heat sealed with glue to one another thereby creating a hollowed circular or spherical shape cover. Once the hollowed absorbent cover 100 has a fragrant latex balloon or other scented hybrid inflatable balloon inflated within the interior confines of the absorbent cover 100, the cover 100 will appear spherical or as a ball. It should be noted that the cover 100 can be designed to create any geometric or non-geometric shape as long as the fragrant latex balloon fully fills the hollowed interior of the shape. In another embodiment, a balloon cover having a non-symmetrical geometric shape can be used along with a custom shaped balloon whereby said shape completely fills the interior cavity of the hallowed textile cover. Also in FIG. 1, a generalized view of the safety flap assembly 101 can be seen, wherein the given scented shaped inflatable balloon can be inserted. More specific information on the unique design elements of the safety flap assembly 101 are provided below.

In an embodiment, the cover 100 can be made of a textile material. Textile materials can generally be classified into three basic categories: woven, non-woven and knit. These three categories further include a far greater variety of all textile materials from synthetics to hybrids to cottons, all of which has a variety of sub-classifications based on specific elemental filament typing etc. To the average consumer the range of classifications of textile materials can be seen by understanding that there are those textiles used for a new born baby to those used by NASA to cover parts of a rover on the planet Mars. However, as a statement of scientific fact in the current context, most any textile or material fabric, natural or synthetic, will generally act to be absorbent, non-absorbent or any combination thereof.

In an embodiment, cotton is the most absorbent and cost effective material that can be used for a scented textile balloon cover. Cotton allows for retention and the slowing release of aromatic vapor, gaseous and/or gas evaporative emission. In an embodiment, a cotton textile known as flannel can also be used as a textile for a fragranced balloon cover. Flannel is naturally thick, absorbent, and generally densely woven providing time-delay and time-release scientific qualities as an absorbent medium for fragrant vapor, gaseous and/or gas evaporative molecules. A dense woven flannel material composed of thicker threads or filaments with a layered matrix pattern being woven at a thickness of approximately 0.025 thousandths of an inch provides an embodiment as a textile material regardless of how much fragrance comes from any varied scented inflatable shaped body. Foil balloons with scented ink or any other applied scented material on the exterior would also become more concentrated within an embodiment textile cover over its surface and time release aromatic fragrant gas into the surrounding environment.

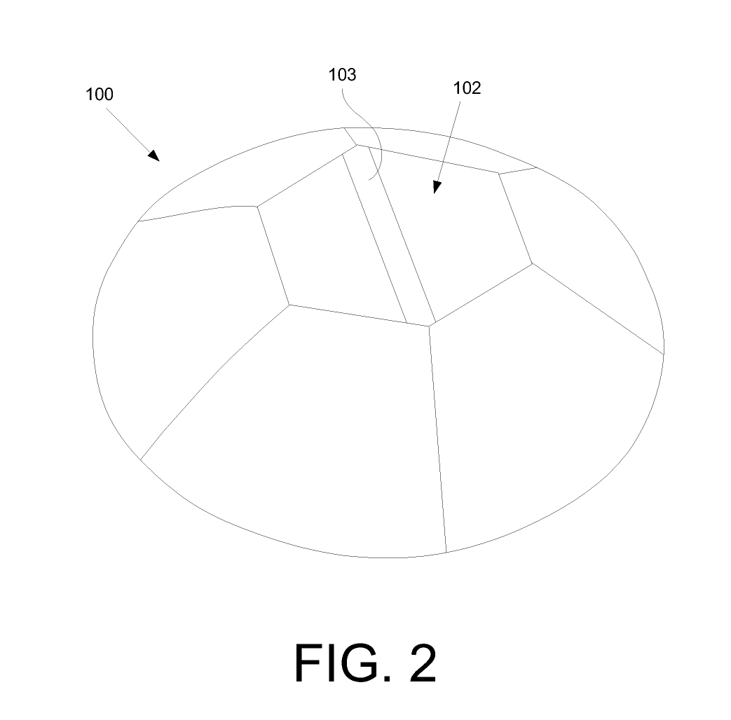

FIG. 2 illustrates a bottom perspective view of the cover illustrated in FIG. 1. FIG. 2 illustrates the closed end 102 of the cover 100. In an embodiment, the closed end 102 is a hexagonal shape and can be made of a singular piece of cut textile material. In this embodiment, there is no possible entry or exit into to cover 100 through the closed end 102. This singular piece of material that constitutes the closed end 102 also acts to enhance the structural strength of the cover 100 as it is under positive expansive pressure from the inflated latex balloon or other elastic fragranced shape which is filled with a pressurized gas thereby creating positive pressure within the balloon thereby pushing the cover seams and shape outward. In an embodiment, a textile or elastic loop 103 can also be included by attaching the loop 103 between the joined seams of the closed end 102 and the cover 100. In an embodiment, the loop 103 can be used to allow the cover 100 to be hung vertically or to be used as an aromatic punching ball.

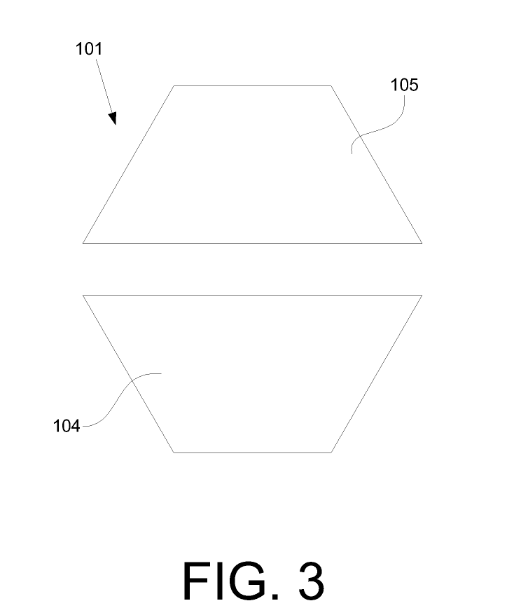

FIG. 3 generally illustrates the two halves 104, 105 that form the safety flap assembly 101 shown in FIG. 1. In an embodiment, flap 105 can be elongated so that flap 105 overlaps flap 104, or vice versa.

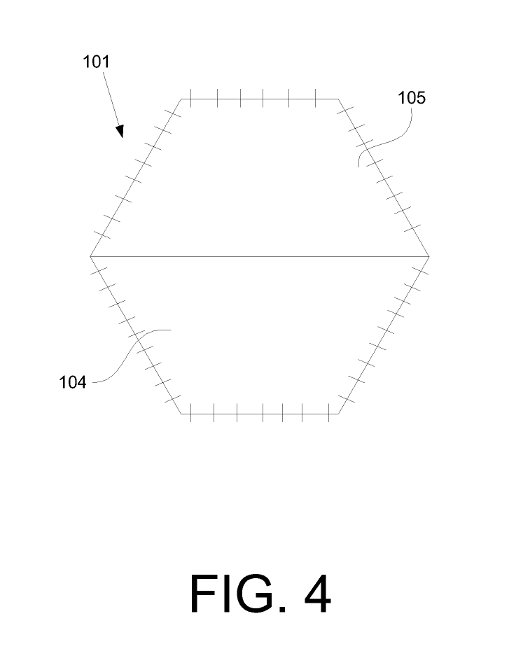

FIG. 4 illustrates an embodiment of safety flap assembly 101 wherein flap 104 and flap 105 have been combined. In this embodiment, the entire perimeter of the two flaps 104, 105 are sewn together forming a singular hexagonal piece. In this embodiment, flaps 104 and 105 are not sewn together along the edge where they meet in order to allow a balloon to be inserted within the cover 100.

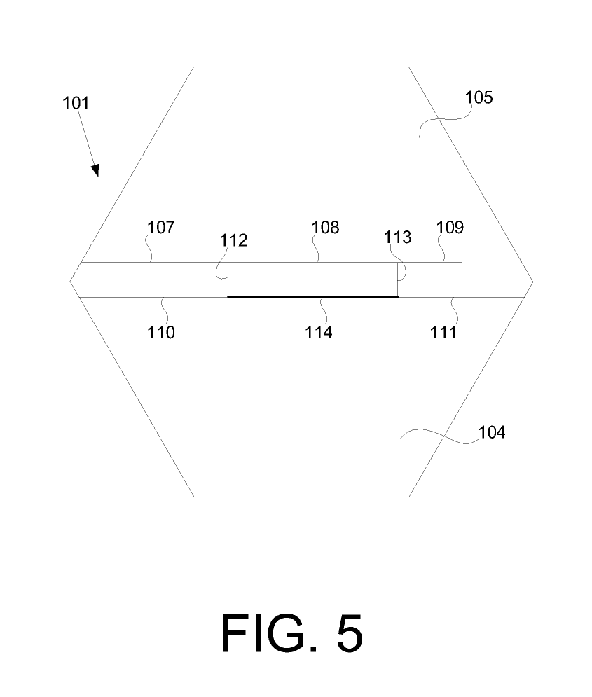

FIG. 5 illustrates an embodiment of the completed safety flap assembly 101 wherein flap 105 can be seen as partially overlapping flap 104. In this embodiment, flap 105 is attached to flap 104 along horizontal lines 107-111 and vertical lines 112 and 113. Horizontal line 114, represents a section of the cover 100 where flap 104 is not attached to flap 105. Accordingly, a slot is created within the safety flap assembly 101 whereby a scented balloon can be inserted into the cover 100.

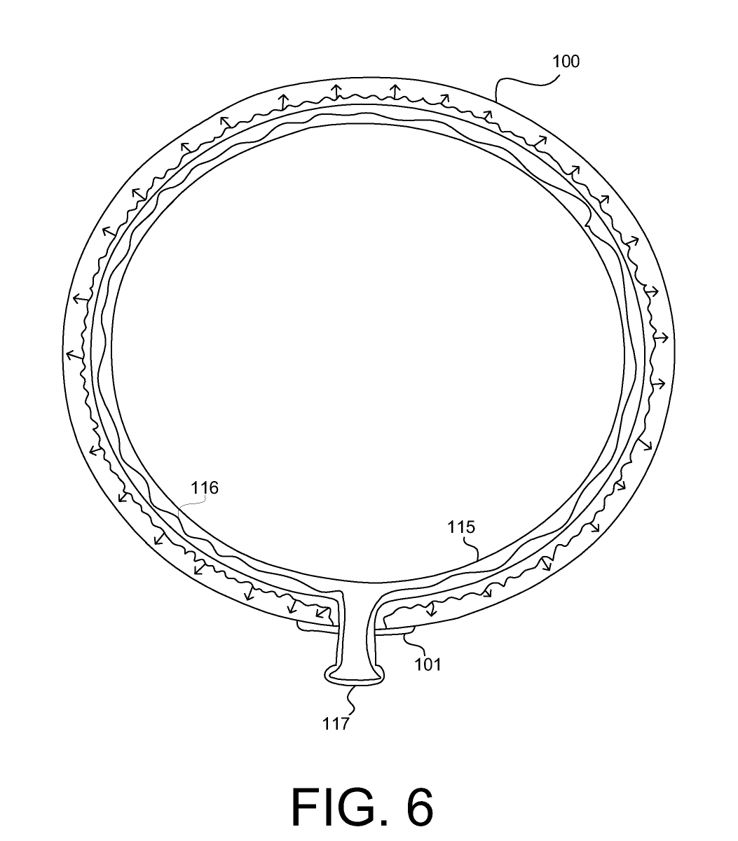

FIG. 6 illustrates a cross-sectional view of an embodiment of an inflated fragranced latex balloon 115 inside cover 100. Methods for providing a fragrance to cover 100 using balloon 115 will be described with respect to FIG. 6.

In an embodiment, a scented balloon 115 can be inserted into an opening within the cover 100 through a slot located in safety flap assembly 101. In this embodiment, balloon 115 is an inflatable balloon made of latex that has been treated with a chemical fragrance. As shown in FIG. 6, the chemical fragrance 116 has been absorbed within the walls of the balloon 115. In an embodiment, the balloon can be treated with the fragrance within the interior portion of the balloon. In another embodiment, the balloon can be treated on the exterior surface of the balloon. In yet another embodiment, the balloon can be manufactured with fragrance in the liquid latex as the balloon paddle is dipped into the warmed liquid latex. The balloon 115 can be treated with a fragrance using any method that would be known to one having ordinary skill in the art. For example, a method of scenting the balloon 115 is described in U.S. patent application Ser. No. 11/866,123, entitled “Interior Chemical Treatments for Inflatable Balloons,” filed on Oct. 2, 2007 by Sean W. Butler, which is incorporated by reference in its entirety.

As the balloon 115 is inserted into the cover 100, the neck or stem 117 of the balloon 115 can extend out of cover 100 through safety flap assembly 101 to allow balloon 115 to be inflated within cover 100. Once the balloon 115 has been inserted into the cover 100, it can be inflated. Once inflated, the balloon neck 117 can be knotted and tucked under the safety flap assembly 101 for safe usage.

It is noted that before inflation, the initially flaccid scented balloon walls are at their thickest state. The typical latex balloon 115 has a wall thickness ranging from approximately 0.005 thousandths of an inch just above the neck 117 and generally increases to 0.008 thousandths of an inch at the top of the bulb or near the drip point. As inflation begins, the thinning fragrance carrying membrane walls of the latex balloon 115 begin to expel the fragrance as a liquid, vapor and/or gas, depending on how saturated the balloon is with the chemical fragrance. As inflation reaches its maximum point as defined by the confines of the interior walls of the cover 100, the chemically fragrant molecules 116 from the balloon 115 membrane walls are expelled into the permeable cover's 100 inner walls. The cover 100 absorbs the fragrance and then over an extended period of time provides an aromatic scent to the surrounding environment. In an embodiment, the latex balloon's 115 wall thickness, can be increased in manufacture to varying amounts with a custom made balloon dramatically increasing the balloon's wall thickness and storage capacity for chemical fragrance which is most often held in the interior of the latex balloon membrane walls.

The exterior of the cover 100 is dry to the human touch and given the time-delayed effect of the textile cover 100, with regard to the migration of chemical fragrance. Ultimately the fragrant gas wafts from the exterior of the cover 100 over an extended period of time as opposed to a plain uncovered scented balloon, which quickly emits a burst of fragrance into the surrounding room and dissipates within a few minutes.

In further comparison, a diffused fragrant spray from a can of air deodorizer is meant to chemically fragrance and mask odors with neutralizing agents which contain specialized chemical components and additionally contain varied fragrances of consumer choice. Some such sprays actually kill air borne and surface bacteria. Still the fragrance of all such atomized sprays last only about twenty minutes and can coat glass or wood tables with a light moist mist. Any containers of liquid drinks in the area can be immediately ruined by the taste of such chemical fragrance as the gaseous mist drops vertically to the floor coating everything in its diffusive path.

In another embodiment of the present invention, a plurality of covers can be used. In an embodiment, a first cover is made of a material that is harder than the second textile cover. In an embodiment, the second cover can be made of pliable perforated foam or a harder absorbent shell, scented or non-scented, with a multiplicity of apertures. In this cover embodiment, the same general scientific means to an end is achieved whereas the interior inflated scented balloons fragrant emission burst is still dramatically diminished due to an estimated +90% of the balloon surface area being covered by the shell embodiment, thereby reducing the surface area of fragrant evaporation as opposed to an uncovered scented balloons immediate 100% burst of fragrance. As with a permeable textile cover, both of the previous structural concepts reduce the exposed surface area of the fragrant balloon acting to limit the amount of fragrance which can be emitted.

As a further explanation of the previously described concept, with the combined area of all the small apertures equalling only about 10% of the total surface area of the cover encasement, 90% of the fragrant latex balloons surface area is not exposed to the surrounding atmosphere in a given room of scientific experimentation. As a result, two additional embodiments both serve to dramatically reduce the rate with which gas evaporates from the exterior of the now aromatized balloon cover thereby creating a similar time-delay design feature. The initial fragrance emission burst is held back within both the absorbent harder shell cover with apertures and the textile cover whereby much of the potential fragrance within the balloon membrane itself remains in the continual process of a fragrance evaporation cycle. The exposed surface area of the apertures is continually replenished by the contained fragrance still within the fragrant permeable body. Essentially the balloon interior breathes in and out over time. The fragrance contained therein starts out as a liquid then vapour and/or gas. In the study of atmospheric sciences, it is similar to a light morning dew or moisture in the ground slowly evaporating with heat and wind. This similar scented balloon scientific event also causes motion and movement within this permeable objects inner hollow cavity which breathes with air and/or object movement with the further evaporation of fragrance finding its way outward through the surface area most exposed with the apertures, i.e. the scented balloon permeable membrane walls. The fragrance is in a continual state of replenishing itself until all the fragrance has evaporated. Therefore both the soft or harder shell permeable balloon cover encasements behave in a similar scientific way to an embodiment of a textile fragranced balloon cover only.

In yet another embodiment, one could put the inflatable fragrant balloon inside the textile balloon cover and in turn insert said two bodies inside a soft or hard shell outer encasement with large holes or slots which would again cause an evaporative time-delay event of fragrance emission into the surrounding environment. The same scientific principles apply as previously described, but here a third harder encasement has been added for different types of play both indoors and outdoors. For example, a scented ball having a configuration like that of a wiffle ball could be used in that one could throw or hit the scented object with a bat outdoors or use indoors to roll on the floor. This ball can also be used in a decorative manner, for example, hanging it from a vertically oriented stand or hanging the ball from a hook under a doorway with a fragrance i.e. mistletoe, once the proper artistic elements were added for the holiday season.

If one simply reverses the outer two encasements positions, as described in the previous paragraph, simple logical reasoning shows a similar but different new fragrant encasement configuration whereby the fragranced balloon is inside the soft or hard shell encasement and is surrounded by a larger textile balloon encasement cover on the exterior, again as a third layer of permeable material, thereby offering a very similar inhibitor design which further delays the evaporative time-delay effects with the partially aromatized textile cover. Imagine a tough and scuff resistant aromatized textile cover over a scented baseball sized object. Even further, one could put a custom re-inflatable balloon inside a PVC plasticized kick ball with very small holes in its surface whereby one could refill air and fragrance into the balloon interior via a customized air and fragrance diffusion tip for reinflation inside the scented balloon which brings pressure to the outer surface of any given sized ball with the balloon being the fragrance driver or carrier of said fragrant material within a pressurized environment for play, amusement or artistic purpose. The fragrance would then evaporate through and out of an outer scuff-resistant textile kick ball cover. Again this version is still yet another embodiment of same scientific and logically derived versions previously described. The end result is still a scented or aromatized object. Again, one can simply reverse the outer two layers or encasements but the fragrance source or driver of the invention, the fragrant or scented inflatable latex balloon, causes the aromatization of the object thereby creating an aromatic medium. On the other, hand an embedded fragrant molded ball, for example, with pieces of fragrant rubber, natural or synthetic, could be the primary fragrance emitter if said fragrant object is covered with a textile or other permeable cover or shell. The results would still all be a function of the strength and volume of the fragrant embedded pieces blow-molded into the surface of the balls body. In this case, the fragrant ball or other formed fragrant shape would be the driver or primary source of the scented emission as an aromatized medium with the textile cover bringing about a stronger concentration of fragrance with its absorptive textile material and still create a time-delayed release of the fragrance from scented printing or the fragranced embedded pieces of rubber in the balls surface covering

While embodiments and applications of this invention have been shown and described, it would be apparent to those skilled in the art, having the benefit of this disclosure that many other embodiment variations and extrapolations than mentioned previously are possible without departing from the inventive concepts set forth herein. The invention, therefore, is not to be restricted except in the spirit of the appended claims.

1. A method of scenting a balloon cover, comprising the steps of:
a) placing an inflatable scented balloon inside at least one cover, wherein a membrane wall of said balloon comprises a fragrance;
b) inflating the balloon within the at least one cover, wherein the balloon transfers the fragrance to the at least one cover; and
c) allowing the at least one cover to absorb the fragrance, thereby providing a scent to the at least one cover and surrounding environment.

2. The method of claim 1, wherein inflating the balloon emits the fragrance as a gas into the interior of the at least cover.

3. The method of claim 1, wherein the balloon transfers a liquid fragrance to the at least one cover.

4. The method of claim 1, wherein the at least one cover is made of a textile material.

5. The method of claim 1, wherein the at least one cover comprises a safety flap assembly adapted to accept and conceal a neck of said balloon.

6. The method of claim 5, wherein the safety flap assembly includes at least two flaps.

7. The method of claim 6, wherein a first flap at least partially overlaps a second flap.

8. The method of claim 1, wherein the at least one cover is made of a plurality of patterned pieces that are connected.

9. The method of claim 1, wherein the balloon is treated with the fragrance on the interior surface of the balloon.

10. The method of claim 1, wherein the balloon is treated with the fragrance on the exterior surface of the balloon.

11. The method of claim 1, wherein the balloon is manufactured with the fragrance in a liquid latex that forms the balloon.

12. The method of claim 1, wherein the balloon comprises one of fragrant inflatable polyurethane and polyvinyl chloride.

13. The method of claim 1, wherein the balloon is made of a foil material.

14. The method of claim 1, wherein the at least one cover includes a plurality of apertures.

15. The method of claim 1 comprising a plurality of covers.

16. The method of claim 15, wherein a first cover is made of a material that is harder than a second cover.

17. The method of claim 1, wherein the at least one cover comprises a loop on an exterior surface of said at least one cover.

18. A balloon cover, comprising:
a) a body adapted to surround a scented inflatable balloon;
b) wherein said body is made of a material that is adapted to absorb a fragrance from the scented inflatable balloon; and
c) wherein said body comprises a safety flap assembly adapted to allow the balloon to be placed inside the cover.
